(19) United States
(12) Patent Application Publication
(10) Pub. No.: US 2004/0176339 A1
(43) Pub. Date: Sep. 9, 2004

(54) Antineoplastic combinations

(75) Inventors: Matthew L. Sherman, Newton, MA (US); James J. Gibbons JR., Westwood, NJ (US); Mark Berger, Merion Station, PA (US); Robert T. Maguire, Doylestown, PA (US); Robert Friedman, Sewell, NJ (US)

Correspondence Address: HOWSON AND HOWSON, CATHY A. KODROFF, ONE SPRING HOUSE CORPORATE CENTER, BOX 457, SPRING HOUSE, PA, 19477, US

(73) Assignee: Wyeth, Madison, NJ

(21) Appl. No.: 10/790,488

(22) Filed: Mar. 1, 2004

Related U.S. Application Data
(60) Provisional application No. 60/452,289, filed on Mar. 5, 2003.

(51) Int. Cl. A61K 31/56

(52) U.S. Cl. 514/171; 514/383; 514/397

(57) ABSTRACT

This invention provides the use of a combination of CCI-779 and an aromatase inhibitor in the treatment of neoplasms.

### CROSS-REFERENCE TO RELATED APPLICATION

This application claims the benefit under 35 USC 119(c) of the priority of U.S. patent application Ser. No. 60/452,289, filed Mar. 5, 2003.

### BACKGROUND OF THE INVENTION

This invention relates to the treatment of neoplasms.

Rapamycin is a macrocyclic triene antibiotic produced by Streptomyces hygroscopicus, which was found to have antifungal activity, particularly against Candida albicans, both in vitro and in vivo [C. Vezina et al., J. Antibiot. 28, 721 (1975); S. N. Sehgal et al., J. Antibiot. 28, 727 (1975); H. A. Baker et al., J. Antibiot. 31, 539 (1978); U.S. Pat. No. 3,929,992; and U.S. Pat. No. 3,993,749]. Additionally, rapamycin alone [U.S. Pat. No. 4,885,171] or in combination with picibanil [U.S. Pat. No. 4,401,653] has been shown to have antitumor activity.

The immunosuppressive effects of rapamycin have been disclosed in FASEB 3, 3411 (1989). Cyclosporin A and FK-506, other macrocyclic molecules, also have been shown to be effective as immunosuppressive agents, therefore useful in preventing transplant rejection [FASEB 3, 3411 (1989); FASEB 3, 5256 (1989); R. Y. Calne et al., Lancet 1183 (1978); and U.S. Pat. No. 5,100,899]. R. Martel et al. [Can. J. Physiol. Pharmacol. 55, 48 (1977)] disclosed that rapamycin is effective in the experimental allergic encephalomyelitis model, a model for multiple sclerosis; in the adjuvant arthritis model, a model for rheumatoid arthritis; and effectively inhibited the formation of IgE-like antibodies.

Rapamycin is also useful in preventing or treating systemic lupus erythematosus [U.S. Pat. No. 5,078,999], pulmonary inflammation [U.S. Pat. No. 5,080,899], insulin dependent diabetes mellitus [U.S. Pat. No. 5,321,009], skin disorders, such as psoriasis [U.S. Pat. No. 5,286,730], bowel disorders [U.S. Pat. No. 5,286,731], smooth muscle cell proliferation and intimal thickening following vascular injury [U.S. Pat. Nos. 5,288,711 and 5,516,781], adult T-cell leukemia/lymphoma [European Patent Application 525,960 A1], ocular inflammation [U.S. Pat. No. 5,387,589], malignant carcinomas [U.S. Pat. No. 5,206,018], cardiac inflammatory disease [U.S. Pat. No. 5,496,832], and anemia [U.S. Pat. No. 5,561,138].

Rapamycin 42-ester with 3-hydroxy-2-(hydroxymethyl)-2-methylpropionic acid (CCI-779) is an ester of rapamycin which has demonstrated significant inhibitory effects on tumor growth in both in vitro and in vivo models. The preparation and use of hydroxyesters of rapamycin, including CCI-779, are disclosed in U.S. Pat. Nos. 5,362,718 and 6,277,983.

CCI-779 exhibits cytostatic, as opposed to cytotoxic properties, and may delay the time to progression of tumors or time to tumor recurrence. CCI-779 is considered to have a mechanism of action that is similar to that of sirolimus. CCI-779 binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from G1 to S. The mechanism of action of CCI-779 that results in the G-S phase block is novel for an anticancer drug.

In vitro, CCI-779 has been shown to inhibit the growth of a number of histologically diverse tumor cells. Central nervous system (CNS) cancer, leukemia (T-cell), breast cancer, prostate cancer, and melanoma lines were among the most sensitive to CCI-779. The compound arrested cells in the G1 phase of the cell cycle.

In vivo studies in nude mice have demonstrated that CCI-779 has activity against human tumor xenografts of diverse histological types. Gliomas were particularly sensitive to CCI-779 and the compound was active in an orthotopic glioma model in nude mice. Growth factor (platelet-derived)-induced stimulation of a human glioblastoma cell line in vitro was markedly suppressed by CCI-779. The growth of several human pancreatic tumors in nude mice as well as one of two breast cancer lines studied in vivo also was inhibited by CCI-779.

### DETAILED DESCRIPTION OF THE INVENTION

This invention provides the use of combinations of CCI-779 and an aromatase inhibitor as antineoplastic combination chemotherapy. In particular, these combinations are useful in the treatment of renal cancer, soft tissue cancer, breast cancer, neuroendocrine tumor of the lung, cervical cancer, uterine cancer, head and neck cancer, glioma, non-small lung cell cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer. This invention also provides combinations of CCI-779 and an aromatase inhibitor for use as antineoplastic combination chemotherapy, in which the dosage of either CCI-779 or the aromatase inhibitor or both are used in subtherapeutically effective dosages. Letrozole is the preferred aromatase inhibitor.

This invention also provides use of combinations of 42-O-(2-hydroxy)ethyl rapamycin and an aromatase inhibitor as antineoplastic combination chemotherapy. The preparation of 42-O-(2-hydroxy)ethyl rapamycin is described in U.S. Pat. No. 5,665,772, which is hereby incorporated by reference.

As used in accordance with this invention, the term “treatment” means treating a mammal having a neoplastic disease by providing said mammal an effective amount of a combination of CCI-779 and an aromatase inhibitor with the purpose of inhibiting growth of the neoplasm in such mammal, eradication of the neoplasm, or palliation of the mammal.

As used in accordance with this invention, the term “providing,” with respect to providing the combination (including simultaneous, separate or sequential administration of the components of the combination), means either directly administering the combination, or administering a prodrug, derivative, or analog of one or both of the components of the combination which will form an effective amount of the combination within the body.

Aromatase is an enzyme which converts androgens to estrone. Estrone can subsequently be converted to estradiol, which has been linked to increased growth or proliferation of estrogen receptor positive carcinoma. As used in accordance with this invention, the term “aromatase inhibitor” means compounds or substances which inhibit the activity of the enzyme aromatase. Thus the goal of using aromatase inhibitors in chemotherapy is typically to reduce the levels of circulating estradiol, to ultimately inhibit the growth of neoplasms that are estrogen receptor positive. There are two types of aromatase inhibitors; steroidal (type I inhibitors) and non-steroidal inhibitors (type II inhibitors). Examples of steroidal aromatase inhibitors include exemestane, formestane, and atamestane, and the like. Examples of non-steroidal aromatase inhibitors include fadrozole, letrozole, vorozole, anastrozole, YM511 [Susaki et al, J. Steroid Biochem Molec Biol, 58:89-194 (1996) and the like. When used with CCI-779 or 42-O-(2-hydroxy)ethyl rapamycin, letrozole is the preferred aromatase inhibitor.

It is also preferred that the combination of CCI-779 and an aromatase inhibitor be used in an treating estrogen receptor positive carcinoma, particularly estrogen receptor positive breast or ovarian cancer.

The preparation of CCI-779 is described in U.S. Pat. No. 5,362,718, which is hereby incorporated by reference. A regiospecific synthesis of CCI-779 is described in U.S. Pat. No. 6,277,983, which is hereby incorporated by reference. Letrozole is commercially available [e.g., as Femara® (Novartis), CGS 20267].

As used in this invention, the combination regimen can be given simultaneously or can be given in a staggered regimen, with CCI-779 being given at a different time during the course of chemotherapy than an aromatase inhibitor. This time differential may range from several minutes, hours, days, weeks, or longer between administration of the two agents. Therefore, the term combination does not necessarily mean administered at the same time or as a unitary dose, but that each of the components are administered during a desired treatment period. The agents may be administered by the same or different routes. For example, one component may be administered orally, while the other parenterally. These combination can be administered daily, weekly, or even once monthly. As typical for chemotherapeutic regimens, a course of chemotherapy may be repeated several weeks later, and may follow the same timeframe for administration of the two agents, or may be modified based on patient response.

The combinations of the invention may be in the form of a kit of parts. The invention therefore includes a product containinig (a) CCI-779 or 42-O-(2-hydroxy)ethyl rapamycin and (b) an aromatase inhibitor as a combined preparation for simultaneous, separate or sequential use in treating a neoplasm in a mammal in need thereof. The invention also includes a pharmaceutical pack containing a course of treatment of a neoplasm for one individual mammal, wherein the pack contains (a) units of CCI-779 or 42-O-(2-hydroxy)ethyl rapamycin in unit dosage form and (b) units of an aromatase inhibitor in unit dosage form.

For the combination of CCI-779 and letrozole, it is preferred that both components are provided orally, and that the initial oral dosage of CCI-779 will in the range of about 2 to about 100 mg/day, 5 mg/day to 75 mg/day, 10 mg/day to 50 mg/day, 15 mg/day to 35 mg/day, or about 20 mg/day to 25 mg/day (on days that it is provided) and the initial oral dose of letrozole will be about 0.1 to 10 mg daily, 0.5 mg to 5 mg, or 1 to 3 mg, or about 2.5 mg (on days that it is provided).

When the combination of CCI-779 and letrozole are provided orally, it is preferred that the CCI-779 and letrozole are provided daily, or that the CCI-779 is provided 5 times every two weeks, while the letrozole is provided daily.

As typical with chemotherapy, dosage regimens are closely monitored by the treating physician, based on numerous factors including the severity of the disease, response to the disease, any treatment related toxicities, age, health of the patient, and other concomitant disorders or treatments. After one or more treatment cycles, the dosages can be adjusted upwards or downwards depending on the results obtained and the side effects observed.

In providing chemotherapy, multiple agents having different modalities of action are typically used as part of a chemotherapy “cocktail.” It is anticipated that the combinations of this invention will be used as part of a chemotherapy cocktail that may contain one or more additional antineoplastic agents depending on the nature of the neoplasia to be treated.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

Particularly suitable oral formulations for rapamycin 42-ester with 3-hydroxy-2-(hydroxymethyl)-2-methylpropionic acid are disclosed in U.S. Ser. No. 60/411,264 and PCT/US03/29228, which are hereby incorporated by reference. Such an oral formulation contains a granulation prepared using a wet granulation process. The granulation contains CCI-779, a water soluble polymer, a pH modifying agent, a surfactant, and an antioxidant. In one embodiment, the formulation contains from 0.1 to 30%, from 0.5 to 25%, from 1 to 20%, from 5 to 15%, or from 7 to 12% (wt/wt) CCI-779, from 0.5 to 50%, from 1 to 40%, from 5 to 35%, from 10 to 25%, or from 15 to 20% (wt/wt) water soluble polymer, from 0.5 to 10%, 1 to 8%, or 3 to 5% (wt/wt) surfactant, and from 0.001% to 1%, 0.01% to 1%, or 0.1% to 0.5% (wt/wt) antioxidant. However, other embodiments may contain more, or less, of these components.

The oral formulation may also contain suitable chelating agents, fillers, binders, surfactants, and the like to facilitate the granulation and tableting process. It is preferred that the wet granulation be performed with a hydroalcoholic solvent system comprising water and an alcohol, with ethanol being the preferred alcoholic component.

Typical water soluble polymers include, but are not limited to, polyvinylpyrrolidone (PVP), hydroxypropylmethylcellulose (HPMC), polyethylene glycol (PEG), and cyclodextrin or mixtures thereof. It is preferred that the water-soluble polymer is PVP, and having a molecular weight of between 2.5 and 60 kilodaltons. Any given oral formulation useful in the invention may contain multiple ingredients of each class of component. For example, an oral formulation containing an antioxidant may contain one or more antioxidants as the antioxidant component.

Acceptable pH modifying agents include, but are not limited to citric acid, sodium citrate, dilute HCl, and other mild acids or bases capable of buffering a solution containing CCI-779 to a pH in the range of about 4 to about 6. Acceptable antioxidants include, but are not limited to, citric acid, d,l-α-tocopherol, BHA, BHT, monothioglycerol, ascorbic acid, and propyl gallate. It is expected that the antioxidants of the oral formulations used in this invention will be used in concentrations ranging from 0.001% to 3% wt/wt. Chelating agents, and other materials capable of binding metal ions, such as ethylene diamine tetra acetic acid (EDTA) and its salts are capable. of enhancing the stability of CCI-779. Surfactants may include polysorbate 80, sodium lauryl sulfate, sodium dodecyl sulfate, salts of bile acids (taurocholate, glycocholate, cholate, deoxycholate, etc.) that may be combined with lecithin. Alternatively, ethoxylated vegetable oils, such as Cremophor EL, vitamin E tocopherol propylene glycol succinate (Vitamin E TGPS), polyoxyethylene-polyoxypropylene block copolymers, and poloxamers. Binders, fillers, and disintegrants such as sucrose, lactose, microcrystalline cellulose, croscarmellose sodium, magnesium stearate, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethycellulose phthalate, noncrystalline cellulose, cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol, and the like may also be incorporated into the oral formulation.

The oral formulation useful in the method of the invention can be prepared by preparing an alcoholic solution comprising CCI-779 and an antioxidant, and an aqueous solution comprising a water-soluble polymer, a surfactant, and a pH modifier, in sufficient quantity to adjust the pH of the aqueous solution to 4 to 6. Suitable alcohols include methanol, ethanol, isopropanol, and the like, where ethanol is the preferred alcohol. The solutions were mixed and added to a mixer containing intragranular excipients. Alternatively, the alcoholic and aqueous solutions can be added separately without mixing with each other. Such intragranular excipients comprise binders and fillers to promote dissolution enhancement. Typical intragranular excipients may include, but are not limited to, microcrystalline cellulose, lactose, and croscarmellose sodium. The solid intragranular excipients are granulated with the solutions in the mixer until a uniform granulation is achieved. The mixer can be a blender with intensifying bar, a low shear granulator or a high shear granulator. The granulation is dried in a fluid bed dryer at approximately 50° C., and milled using a suitable milling device, such as a Fitz mill. The wet granulation and drying can be done in a fluid bed granulator/dryer. The wet granulation can be dried using a tray drying oven. If desired, the dried granulation can be further blended with extragranular fillers and binders, such as microcrystalline cellulose, croscarmellose sodium, and magnesium stearate in a blender, such as a V-blender, before compression into tablets.

Alternatively, some of the water-soluble polymer can be contained in the intragranular excipients, and the aqueous and alcoholic solutions added to the mixer containing the intragranular excipients stepwise. For example, the order of addition to the mixer may be one half of the aqueous solution, followed by the entire alcoholic solution, and then the remainder of the aqueous solution. Other sequences of addition are possible and permissible in these solid oral formulations.

In some cases it may be desirable to administer the compounds directly to the airways in the form of an aerosol.

The compounds may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Particularly suitable injectable formulations for rapamycin 42-ester with 3-hydroxy-2-(hydroxymethyl)-2-methylpropl acid are disclosed in U.S. patent application Ser. No. 10/626,943 and PCT/US03/223276, which are hereby incorporated by reference. In this embodiment, the injectable formulation useful in the invention provides a CCI-779 cosolvent concentrate containing an parenterally acceptable solvent and an antioxidant as described above and a parenteral formulation containing CCI-779, composed of CCI-779, an parenterally acceptable cosolvent, an antioxidant, a diluent solvent, and a surfactant. Any given formulation useful in this invention may contain multiple ingredients of each class of component. For example, a parenterally acceptable solvent can include a non-alcoholic solvent, an alcoholic solvent, or mixtures thereof. Examples of suitable non-alcoholic solvents include, e.g., dimethylacetamide, dimethylsulfoxide or acetonitrile, or mixtures thereof. “An alcoholic solvent,” may contain one or more alcohols as the alcoholic solvent component of the formulation. Examples of solvents useful in the formulations invention include, without limitation, ethanol, propylene glycol, polyethylene glycol 300, polyethylene glycol 400, polyethylene glycol 600, polyethylene glycol 1000, or mixtures thereof. These cosolvents are particularly desirable because degradation via oxidation and lactone cleavage occurs to a lower extent for these cosolvents. Further, ethanol and propylene glycol can be combined to produce a less flammable product, but larger amounts of ethanol in the mixture generally result in better chemical stability. A concentration of 30 to 100% v/v of ethanol in the mixture is preferred.

In this embodiment, the stability of CCI-779 in parenterally acceptable alcoholic cosolvents is enhanced by addition of an antioxidant to the formulation. Acceptable antioxidants include, but are not limited to, citric acid, d,l-α-tocopherol, BHA, BHT, monothioglycerol, ascorbic acid, propyl gallate, and mixtures thereof. Generally, the parenteral formulations useful in this embodiment of the invention will contain an antioxidant component(s) in a concentration ranging from 0.001% to 1% w/v, or 0.01% to 0.5% w/v, of the cosolvent concentrate, although lower or higher concentrations may be desired. Of the antioxidants, d,l-α-tocopherol is particularly desirable and is used at a concentration of 0.01 to 0.1% w/v with a preferred concentration of 0.075% w/v of the cosolvent concentrate.

In certain embodiments, the antioxidant component of the formulation of the invention also exhibits chelating activity. Examples of such chelating agents include, e.g., citric acid, acetic acid, and ascorbic acid (which may function as both a classic antioxidant and a chelating agent in the present formulations). Other chelating agents include such materials as are capable of binding metal ions in solution, such as ethylene diamine tetra acetic acid (EDTA), its salts, or amino acids such as glycine are capable of enhancing the stability of CCI-779. In some embodiments, components with chelating activity are included in the formulations of the invention as the sole “antioxidant component”. Typically, such metal-binding components, when acting as chelating agents are used in the lower end of the range of concentrations for the antioxidant component provided herein. In one example, citric acid enhanced the stability of CCI-779 when used at a concentration of less than 0.01% w/v. Higher concentrations are less stable solutions and thus, less desirable for products to be subject to long-term storage in liquid form. Additionally, such chelating agents may be used in combination with other antioxidants as part of the antioxidant component of the invention. For example, an acceptable formulation may contain both citric acid and d,l-α-tocopherol. Optimal concentrations for the selected antioxidant(s) can be readily determined by one of skill in the art, based upon the information provided herein.

Advantageously, in certain embodiments of the parenteral formulations useful in the invention, precipitation of CCI-779 upon dilution with aqueous infusion solutions or blood is prevented through the use of a surfactant contained in the diluent solution. The most important component of the diluent is a parenterally acceptable surfactant. One particularly desirable surfactant is polysorbate 20 or polysorbate 80. However, one of skill in the art may readily select other suitable surfactants from among salts of bile acids (taurocholate, glycocholate, cholate, deoxycholate, etc.) which are optionally combined with lecithin. Alternatively, ethoxylated vegetable oils, such as a pegylated castor oil [e.g., such as PEG-35 castor oil which is sold, e.g., under the name Cremophor EL, BASF], vitamin E tocopherol propylene glycol succinate (Vitamin E TGPS), and polyoxyethylene-polyoxypropylene block copolymers can be used in the diluent as a surfactant, as well as other members of the polysorbate family such as polysorbate 20 or 60 Other components of the diluent may include water, ethanol, polyethylene glycol 300, polyethylene 400, polyethylene 600, polyethylene 1000, or blends containing one or more of these polyethylene glycols, propylene glycol and other parenterally acceptable cosolvents or agents to adjust solution osmolarity such as sodium chloride, lactose, mannitol or other parenterally acceptable sugars, polyols and electrolytes. It is expected that the surfactant will comprise 2 to 100% w/v of the diluent solution, 5 to 80% w/v, 10 to 75% w/v, 15 to 60% w/v, and preferably, at least 5% w/v, or at least 10% w/v, of the diluent solution.

A parenteral formulation useful in the invention can be prepared as a single solution, or preferably can be prepared as a cosolvent concentrate containing CCI-779, an alcoholic solvent, and an antioxidant, which is subsequently combined with a diluent that contains a diluent solvent and suitable surfactant. Prior to use, the cosolvent concentrate is mixed with a diluent comprising a diluent solvent, and a surfactant. When CCI-779 is prepared as a cosolvent concentrate according to this invention, the concentrate can contain concentrations of CCI-779 from 0.05 mg/mL, from 2.5 mg/mL, from 5 mg/mL, from 10 mg/mL or from 25 mg/mL up to approximately 50 mg/ml. The concentrate can be mixed with the diluent up to approximately 1 part concentrate to 1 part diluent, to give parenteral formulations having concentrations of CCI-779 from 1 mg/mL, from 5 mg/mL, from 10 mg/mL, from 20 mg/mL, up to approximately 25 mg/ml. For example the concentration of CCI-779 in the parenteral formulation may be from about 2.5 to 10 mg/mL. This invention also covers the use of formulations having lesser concentrations of CCI-779 in the cosolvent concentrate, and formulations in which one part of the concentrate is mixed with greater than 1 part of the diluent, e.g., concentrate: diluent in a ratio of about 1:1.5, 1:2, 1:3, 1:4, 1:5, or 1:9v/v and so on, to CCI-779 parenteral formulations having a CCI-779 concentration down to the lowest levels of detection.

Typically the antioxidant may comprise from about 0.0005 to 0.5% w/v of the formulation. The surfactant may for example comprise from about 0.5% to about 10% w/v of the formulation. The alcoholic solvent may for example comprise from about 10% to about 90% w/v of the formulation.

The parenteral formulations useful in this invention can be used to produce a dosage form that is suitable for administration by either direct injection or by addition to sterile infusion fluids for intravenous infusion.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The following examples are illustrative of the present invention, but are not a limitation thereof.

### EXAMPLE 1

CCI-779 in combination with an aromatase inhibitor in a neoplasm

The combination of CCI-779 and letrozole in postmenopausal women with locally advanced or metastatic breast cancer is being evaluated in this clinical trial.

Fifty-five patients (pts) were enrolled. Randomization is in a 1:1:1 ratio (˜30 evaluable pts/arm), letrozole alone: letrozole with CCI daily (CCI daily arm): letrozole with CCI daily for 5 days every 2 weeks (CCI intermittent arm). All pts receive 2.5 mg letrozole daily.

Initially, 6 patients each were enrolled at high dose (HD) schedules, 25 mg CCI daily and 75 mg CCI intermittent; 3 patients in each arm had toxicity that resulted in dose delay/reduction or discontinuation. Thus, the protocol was amended and doses were reduced to low dose (LD) schedules, 10 mg CCI daily and 30 mg CCI intermittent. As of 1 Dec. 2003, 12 and 23 patients were enrolled in the HD and LD schedules, respectively. The median age was 60 yrs (range, 42-81). Safety data are available for 12 pts treated with the HD schedules (25 mg, 6 pts; 75 mg, 6 pts), 11 pts treated with the LD schedules (10 mg, 4 pts; 30 mg, 7 pts), and 12 pts treated with letrozole alone. The most frequently occurring grade 3-4 CCI related toxicity was stomatitis for the HD schedules (2/6 pts, 1/6 pts) and diarrhea for the LD schedules (0 pts, 1/7 pts). No grade 3-4 toxicities were reported for pts treated with letrozole alone. Of 55 pts, 7 have been on study for 40+ wk. Preliminary tumor responses (RECIST) are available for 19 evaluable pts. CCI pts (n=13) had 1 complete response (HD schedule), 3 partial responses (HD schedules), 9 stable disease (6 pts on HD schedules, 3 on LD schedules, incl 4 pts on HD schedules with SD 24 wk). Letrozole-alone pts (n=6) had 2 PR and 4 SD (including 1 pt with SD 24 wk)

The combination of 10 mg CCI daily or 30 mg CCI intermittent with letrozole showed favorable results for tolerability.

### EXAMPLE 2

Tablets each containing 2.5 mg of letrozole and also tablets each containing a dose of CCI-779 as mentioned in Example 1 are packaged in a container to provide a course of treatment for a patient.

All patents, patent applications, articles, and other documents referenced herein are incorporated by reference. It will be clear to one of skill in the art that modifications can be made to the specific embodiments described herein without departing from the scope of the invention.

What is claimed is:

1. A method of treating a neoplasm in a mammal in need thereof, which comprises providing to said mammal an effective amount of a combination comprising CCI-779 and an aromatase inhibitor.

2. The method according to claim 1, wherein the aromatase inhibitor is selected from the group consisting of exemestane, formestane, atamestane, fadrozole, letrozole, vorozole, and anastrozole.

3. The method according to claim 2, wherein the aromatase inhibitor is letrozole.

4. The method according to claim 1, wherein the neoplasm is renal cancer.

5. The method according to claim 1, wherein the neoplasm is soft tissue sarcoma.

6. The method according to claim 1, wherein the neoplasm is breast cancer.

7. The method according to claim 1, wherein the neoplasm is a neuroendocrine tumor of the lung.

8. The method according to claim 1, wherein the neoplasm is cervical cancer.

9. The method according to claim 1, wherein the neoplasm is uterine cancer.

10. The method according to claim 1, wherein the neoplasm is a head and neck cancer.

11. The method according to claim 1, wherein the neoplasm is glioma.

12. The method according to claim 1, wherein the neoplasm is non-small cell lung cancer.

13. The method according to claim 1, wherein the neoplasm is prostate cancer.

14. The method according to claim 1, wherein the neoplasm is pancreatic cancer.

15. The method according to claim 1, wherein the neoplasm is lymphoma.

16. The method according to claim 1, wherein the neoplasm is melanoma.

17. The method according to claim 1, wherein the neoplasm is small cell lung cancer.

18. The method according to claim 1, wherein the neoplasm is ovarian cancer.

19. The method according to claim 1, wherein the neoplasm is colon cancer.

20. The method according to claim 1, wherein the neoplasm is esophageal cancer.

21. The method according to claim 1, wherein the neoplasm is gastric cancer.

22. The method according to claim 1, wherein the neoplasm is leukemia.

23. The method according to claim 1, wherein the neoplasm is colorectal cancer.

24. The method according to claim 1, wherein the neoplasm is unknown primary cancer.

25. A method of treating a neoplasm in a mammal in need thereof, which comprises providing to said mammal an effective amount of a combination comprising CCI-779 and an aromatase inhibitor, wherein either CCI-779, the aromatase inhibitor, or both are provided in subtherapeutically effective amounts.

26. The method according to claim 25 in which CCI-779 is provided in a subtherapeutically effective amount.

27. The method according to claim 25 in which the aromatase inhibitor is provided in a subtherapeutically effective amount.

28. The method according to claim 25 in which both CCI-779 and the aromatase inhibitor are provided in subtherapeutically effective amounts.

29. The method according to claim 25, wherein the aromatase inhibitor is letrozole.

30. An antineoplastic combination comprising an antineoplastic effective amount of a combination of CCI-779 and an aromatase inhibitor.

31. A method of treating a neoplasm in a mammal in need thereof, comprising providing to said mammal an effective amount of a combination comprising 42-O-(2-hydroxy)ethyl rapamycin and an aromatase inhibitor.

32. A method of treating an estrogen receptor positive carcinoma in a mammal in need thereof, comprising providing to said mammal an effective amount of a combination comprising CCI-779 and an aromatase inhibitor.

33. The method according to claim 32, wherein the aromatase inhibitor is selected from the group consisting of exemestane, formestane, atamestane, fadrozole, letrozole, vorozole, and anastrozole.

34. The method according to claim 33, wherein the aromatase inhibitor is letrozole.

35. The method according to claim 32, wherein the estrogen receptor positive carcinoma is of the breast cancer or ovarian cancer.

36. The method according to claim 35, wherein the aromatase inhibitor is letrozole.

37. The method according to claim 32, wherein the CCI-779 or the aromatase inhibitor, or both are provided in subtherapeutically effective amounts.

38. A method of treating an estrogen receptor positive carcinoma in a mammal in need thereof, comprising providing to said mammal an effective amount of a combination comprising 42-O-(2-hydroxy)ethyl rapamycin and an aromatase inhibitor.

39. A product containing (a) CCI-779 or 42-O-(2-hydroxy)ethyl rapamycin and (b) an aromatase inhibitor as a combined preparation for simultaneous, separate or sequential use in treating a neoplasm in a mammal in need thereof.

40. The product according to claim 39, wherein the aromatase inhibitor is selected from the group consisting of exemestane, formestane, atamestane, fadrozole, letrozole, vorozole, and anastrozole.

41. The product according to claim 40, wherein the aromatase inhibitor is letrozole.

42. A pharmaceutical pack containing a course of treatment of a neoplasm for one individual mammal, wherein the pack contains (a) units of CCI-779 or 42-O-(2-hydroxy)ethyl rapamycin in unit dosage form and (b) units of an aromatase inhibitor in unit dosage form.

43. A pharmaceutical pack according to claim 42, wherein the aromatase inhibitor is selected from the group consisting of exemestane, formestane, atamestane, fadrozole, letrozole, vorozole, and anastrozole.

44. A pharmaceutical pack according to claim 42, wherein the aromatase inhibitor is letrozole.

45. A pharmaceutical composition useful in treating a neoplasm in a mammal in need thereof, the composition comprising (a) CCI-779 or 42-O-(2-hydroxy)ethyl rapamycin and (b) an aromatase inhibitor in combination or association with a pharmaceutically acceptable carrier.

46. The pharmaceutical composition according to claim 45, wherein the aromatase inhibitor is selected from the group consisting of exemestane, formestane, atamestane, fadrozole, letrozole, vorozole, and anastrozole.

47. The pharmaceutical composition according to claim 46, wherein the aromatase inhibitor is letrozole.

48. An antineoplastic combination comprising an antineoplastic effective amount of a combination of 42-O-(2-hydroxy)ethyl rapamycin and an aromatase inhibitor.
